(19) United States
(12) Patent Application Publication
(10) Pub. No.: US 2009/0282582 A1
(43) Pub. Date: Nov. 12, 2009

(54) ENVIRONMENTAL STRESS RESPONSIVE PROMOTER AND METHOD OF TISSUE-SPECIFIC GENE EXPRESSION USING THE SAME

(75) Inventors: Motoaki Seki, Kanagawa (JP); Jong-Myong Kim, Kanagawa (JP); Kazuo Shinozaki, Kanagawa (JP); Miki Fujita, Kanagawa (JP)

Correspondence Address: BIRCH STEWART KOLASCH & BIRCH, PO BOX 747, FALLS CHURCH, VA, 22040-0747, US

(73) Assignee: RIKEN, WAKO-SHI SAITAMA (JP)

(21) Appl. No.: 12/311,900

(22) PCT Filed: Oct. 19, 2007
(86) PCT No.: PCT/JP2007/070867

§ 371 Date: Apr. 17, 2009

(30) Foreign Application Priority Data
Oct. 20, 2006 (JP) 2006-286326

(51) Int. Cl. C12N 15/82; C07H 21/04; C12N 15/63; A01H 5/00; C12N 5/10

(52) U.S. Cl. 800/287; 536/24.1; 435/320.1; 800/295; 435/419

(57) ABSTRACT

This invention concerns induction of gene expression specifically in given tissue with the use of a novel promoter that has environmental stress responsiveness and functions specifically in given tissue. It is realized by a method comprising: a step of preparing a plant having an arbitrary gene downstream of the environmental stress responsive promoter comprising DNA (a), (b), or (c) below; (a) DNA comprising the nucleotide sequence as shown in any of SEQ ID NO: 1 to 8; (b) DNA comprising a nucleotide sequence derived from the nucleotide sequence as shown in any of SEQ ID NO: 1 to 8 by deletion, substitution, or addition of one or a plurality of nucleotides and functioning as an environmental stress responsive promoter; or (c) DNA hybridizing under stringent conditions to DNA comprising the nucleotide sequence as shown in any of SEQ ID NO: 1 to 8 and functioning as an environmental stress responsive promoter; and a step of cultivating the plant in the presence of environmental stress, the method comprising inducing expression of the gene located downstream of the environmental stress responsive promoter in a tissue-specific manner.

### TECHNICAL FIELD

The present invention relates to an environmental stress responsive promoter and a method of tissue-specific gene expression using the same.

### BACKGROUND ART

Plant growth is significantly affected by environmental stresses, such as dehydration, high salt concentration, and low temperature stresses. Among these stresses, dehydration or water deficit is the most severe restriction factor for plant growth and crop production. Dehydration stress induces plants to generate various types of biochemical and physiological responses.

Non-Patent Documents 1 and 2 disclose identification of promoters that induce expression when plant bodies are exposed to such environmental stresses with the use of full-length cDNA microarrays. Patent Documents 1 to 4 also disclose various types of environmental stress responsive promoters or disease stress responsive promoters.

However, a finding regarding tissues in which such environmental stress responsive promoters are capable of functioning has not yet been obtained, and promoters that have environmental stress responsiveness and have the capacity for induction of tissue-specific expression have not yet been reported.

Non-Patent Document 1: Seki, M., Narusaka, M., Ishida, J., Nanjo, T., Fujita, M., Oono, Y., Kamiya, A., Nakajima, M., Enju, A., Sakurai, T., Satou, M., Akiyama, K., Taji, T., Yamaguchi-Shinozaki, K., Carninci, P., Kawai, J., Hayashizaki, Y., Shinozaki, K., 2002, Monitoring the expression profiles of 7000 Arabidopsis genes under drought, cold, and high-salinity stresses using a full-length cDNA microarray, Plant, J., 31: 279-292

Non-Patent Document 2: Seki, M., Narusaka, M., Abe, H., Kasuga, M., Yamaguchi-Shinozaki, K., Carninci, P., Hayashizaki, Y., Shinozaki, K., 2001, Monitoring the expression pattern of 1300 Arabidopsis genes under drought and cold stresses using a full-length cDNA microarray, Plant Cell, 13: 61-72

Patent Document 1: JP Patent Application No. 2004-161313

Patent Document 2: JP Patent Application No. 2001-309984

Patent Document 3: U.S. patent Ser. No. 10/495,918

Patent Document 4: JP Patent Application No. 2002-095389

### DISCLOSURE OF THE INVENTION

The present invention is intended to provide a novel promoter that has environmental stress responsiveness and functions specifically in given tissue. The present invention is also intended to provide a method for inducing gene expression specifically in given tissue using such promoter.

The present invention, which has attained the above objects, includes the following.

(1) An environmental stress responsive promoter comprising DNA (a), (b), or (c) below:

(a) DNA comprising the nucleotide sequence as shown in any of SEQ ID NO: 1 and 4 to 8;

(b) DNA comprising a nucleotide sequence derived from the nucleotide sequence as shown in any of SEQ ID NO: 1 and 4 to 8 by deletion, substitution, or addition of one or a plurality of nucleotides and functioning as an environmental stress responsive promoter; or

(c) DNA hybridizing under stringent conditions to DNA comprising the nucleotide sequence as shown in any of SEQ ID NO: 1 and 4 to 8 and functioning as an environmental stress responsive promoter.

(2) The promoter according to (1), wherein the environmental stress is at least one stress selected from the group consisting of low temperature stress, dehydration stress, and salt stress.

(3) The promoter according to (1), which functions in stem and leaf tissue and/or root tissue.

(4) An expression vector comprising the promoter according to (1).

(5) An expression vector comprising an arbitrary gene incorporated into the expression vector according to (4).

(6) A transformant comprising the expression vector according to (4) or (5).

(7) A transgenic plant comprising the expression vector according to (4) or (5).

(8) The transgenic plant according to (7), wherein the plant is a plant body, a plant organ, plant tissue, or cultured plant cells.

(9) A method for producing a stress tolerant plant comprising culturing or cultivating the transgenic plant according to (7) or (8).

(10) A method of tissue-specific gene expression comprising:

a step of preparing a plant having an arbitrary gene downstream of the environmental stress responsive promoter comprising DNA (a), (b), or (c) below;

(a) DNA comprising the nucleotide sequence as shown in any of SEQ ID NO: 1 to 8;

(b) DNA comprising a nucleotide sequence derived from the nucleotide sequence as shown in any of SEQ ID NO: 1 to 8 by deletion, substitution, or addition of one or a plurality of nucleotides and functioning as an environmental stress responsive promoter; or

(c) DNA hybridizing under stringent conditions to DNA comprising the nucleotide sequence as shown in any of SEQ ID NO: 1 to 8 and functioning as an environmental stress responsive promoter; and

a step of cultivating the plant in the presence of environmental stress,

the method comprising inducing expression of the gene located downstream of the environmental stress responsive promoter in a tissue-specific manner.

(11) The method of tissue-specific gene expression according to (10), wherein the environmental stress is at least one stress selected from the group consisting of low temperature stress, dehydration stress, and salt stress.

(12) The method of tissue-specific gene expression according to (10), wherein the gene is induced to express specifically in stem and leaf tissue and/or root tissue.

(13) The method of tissue-specific gene expression according to (10), which further comprises a step of introducing an expression cassette comprising the above gene located downstream of the environmental stress responsive promoter into the plant.

This description includes part or all of the contents as disclosed in the description and/or drawings of Japanese Patent Application No. 2006-286326, which is a priority document of the present application.

### BRIEF DESCRIPTION OF THE DRAWINGS

FIG. 1 is a photograph showing the results of luciferase assay using a promoter of At1g01470 (RAFL05-17-B13) and an LUC transgenic plant.

FIG. 2 is a photograph showing the results of luciferase assay using a promoter of At2g47770 (RAFL05-18-I12) and an LUC transgenic plant.

FIG. 3 is a photograph showing the results of luciferase assay using a promoter of At2g46680 (ATHB-7, RAFL05-20-M16) and an LUC transgenic plant.

FIG. 4 is a photograph showing the results of luciferase assay using a promoter of At3g11410 (RAFL06-07-B19) and an LUC transgenic plant.

FIG. 5 is a photograph showing the results of luciferase assay using a promoter of At2g06050 (RAFL06-16-J10) and an LUC transgenic plant.

FIG. 6 is a photograph showing the results of luciferase assay using a promoter of At2g26530 (RAFL07-08-I12) and an LUC transgenic plant.

FIG. 7 is a photograph showing the results of luciferase assay using a promoter of At4g20830 (RAFL09-07-M01) and an LUC transgenic plant.

FIG. 8 is a photograph showing the results of luciferase assay using a promoter of At2g29450 (RAFL08-17-O07) and an LUC transgenic plant.

### BEST MODES FOR CARRYING OUT THE INVENTION

Hereafter, the present invention is described in greater detail with reference to the drawings.

### 1. Environmental Stress Responsive Promoter

The environmental stress responsive promoter of the present invention has a function of transcribing a gene located at a site downstream when environmental stress is applied. The term “function(ing) as an environmental stress responsive promoter” used herein refers to a function of RNA polymerase to bind to a promoter to initiate transcription when the promoter is exposed to given environmental stress.

The term “environmental stress” generally refers to nonbiological stress, and examples of such stress include dehydration stress, low temperature stress, and high salt concentration stress. The term “dehydration” refers to a condition of moisture deficit. The term “low temperature” refers to the condition resulting from exposure of an organism species to temperature that is lower than its optimal living temperature (e.g., when Arabidopsis thaliana is continuously exposed to temperature at −20° C. to +21° C. for 1 hour to several weeks). The term “high salt concentration” refers to the condition resulting from continuous treatment with 50 mM to 600 mM NaCl for 0.5 hours to several weeks. Such environmental stress may be applied singly or in combinations of two or more.

The environmental stress responsive promoter of the present invention, in particular, is isolated from 8 genes selected from the dehydration, low temperature, or salt stress inducible genes identified in Seki et al., 2002, Plant Journal, 31: 279-292. Specifically, the 8 genes shown in Table 1 were selected.

TABLE 1

| | AGI (MIPS) | |
|---|---|---|
| cDNA clones | code | Description |
| RAFL05-17-B13 | At1g01470 | LEA protein |
| RAFL05-18-I12 | At2g47770 | Homolog of benzodiazepine receptor |
| RAFL05-20-M16 | At2g46680 | ATHB7 transcription factor |
| RAFL06-07-B19 | At3g11410 | AHG3 gene |
| RAFL06-16-J10 | At2g06050 | 12-oxophytodienoate reductase (OPR3) |
| | | gene |
| RAFL07-08-I12 | At2g26530 | Homolog of calmodulin-binding protein |
| RAFL09-07-M01 | At4g20830 | Protein containing FAD-binding domain |
| RAFL08-17-O07 | At2g29450 | Glutathione S-transferase |

The environmental stress responsive promoter of the present invention is a cis-element located upstream of the above 8 genes, and such promoter binds to a transcription factor and activates transcription of the gene located downstream thereof. The promoter region is determined with the use of a gene analysis program by analyzing the nucleotide sequences of the above genes based on the genome information stored in the database (GenBank/EMBL, ABRC). Specifically, the determined nucleotide sequences are shown in SEQ ID NOs: 1 to 8 as examples of the environmental stress responsive promoter of the present invention. Table 2 shows the names of the isolated clones of the promoter, the types of environmental stresses to which the promoter responds, and the relevant sequence numbers.

TABLE 2

| cDNA clones | Environmental stress type | SEQ ID NO: |
|---|---|---|
| RAFL05-17-B13 | Dehydration and salt | SEQ ID NO: 1 |
| RAFL05-18-I12 | Dehydration and salt | SEQ ID NO: 2 |
| RAFL05-20-M16 | Dehydration and salt | SEQ ID NO: 3 |
| RAFL06-07-B19 | Dehydration, salt, | SEQ ID NO: 4 |
| | and low temperature | |
| RAFL06-16-J10 | Dehydration and salt | SEQ ID NO: 5 |
| RAFL07-08-I12 | Dehydration and salt | SEQ ID NO: 6 |
| RAFL09-07-M01 | Dehydration and salt | SEQ ID NO: 7 |
| RAFL08-17-O07 | Dehydration and salt | SEQ ID NO: 8 |

As long as the promoter of the present invention functions as an environmental stress responsive promoter, clones may each have a nucleotide sequence derived from the nucleotide sequence as shown in any of SEQ ID NO: 1 to 8 by deletion, substitution, or addition of 1 or a plurality of, and preferably 1 or several (e.g., 1 to 10 or 1 to 5) nucleotides. Further, the promoter of the present invention comprises DNA hybridizing under stringent conditions to DNA comprising the nucleotide sequence as shown in any of SEQ ID NO: 1 to 8 and functioning as an environmental stress responsive promoter. Under stringent conditions, hybridization is carried out at a sodium concentration of 25 to 500 mM, and preferably 25 to 300 mM, and temperature of 42 to 68° C., and preferably 42 to 65° C. More specifically, hybridization is carried out at 5×SSC (83 mM NaCl, 83 mM sodium citrate) and 42° C.

Examples of methods for introducing variation into the promoter sequence include known methods, such as the Kunkel method and the Gapped duplex method, or methods in accordance therewith. For example, variation is introduced using a kit for introducing variation which utilizes site-directed mutagenesis (e.g., Mutant-K (Takara) or Mutant-G (Takara)) or the LA PCR in vitro Mutagenesis Series Kit (Takara).

The plant promoter of the present invention includes a promoter comprising a nucleotide sequence derived from any of the nucleotide sequences as shown in SEQ ID NOs: 1 to 8 by addition of a nucleotide sequence for enhancing translation efficiency to the 3′ terminus or by deleting the 5′ terminus without sacrificing the promoter activity.

Once the nucleotide sequence of the promoter of the present invention is established, the promoter of the present invention can then be obtained by chemical synthesis, PCR using a cloned probe as a template, or hybridization using a DNA fragment having the nucleotide sequence as a probe. Further, a variant of the promoter of the present invention having functions equivalent to those before the variation can be synthesized by site-directed mutagenesis, etc.

### 2. Construction of Expression Vector

The expression vector of the present invention can be obtained by ligating (inserting) the promoter of the present invention into a suitable vector. Vectors for inserting the promoter of the present invention are not particularly limited, as long as they can replicate the gene of interest in the host. Examples thereof include a plasmid, a shuttle vector, and a helper plasmid.

Examples of plasmid DNAs include: Escherichia coli-derived plasmids, such as pBR322, pBR325, pUC118, pUC119, pUC18, pUC19, and pBluescript; Bacillus subtilis-derived plasmids, such as pUB110 and pTP5; and yeast-derived plasmids, such as YEp13 and YCp50. Examples of phage DNA include λ phages, such as Charon 4A, Charon 21A, EMBL3, EMBL4, λgt10, λgt11, and λZAP. Further, vectors of animal viruses such as retroviruses or vaccinia viruses or vectors of insect viruses such as baculoviruses can be used.

In order to insert the promoter of the present invention into a vector, a method is employed in which the purified DNA is first cleaved with a suitable restriction enzyme and then inserted into the restriction enzyme site or multicloning site of a suitable vector DNA, thereby ligating the promoter to a vector.

In order to express an arbitrary gene, in the present invention, such arbitrary gene can be inserted into the expression vector. Such arbitrary gene can be inserted in the same manner as in the case of insertion of a promoter into a vector. An arbitrary gene is not particularly limited, and an example thereof is a gene encoding a protein that can impart environmental stress tolerance to a plant.

A reporter gene, for example, the GUS gene that is extensively used for plants, may be ligated to the 3′ terminus of the promoter of the present invention, so that the strength of the promoter can be easily evaluated by inspecting GUS activity. In addition to the GUS gene, luciferase, green fluorescent protein, and the like can be used as reporter genes.

Thus, various types of vectors can be used in the present invention. Further, the target arbitrary gene may be ligated to the promoter of the present invention in the sense or antisense direction, and the resultant may be inserted into a binary vector, such as a pBI101 vector (Clonetech).

### 3. Preparation of Transformant

The transformant of the present invention can be obtained by introducing the expression vector of the present invention into a host. Hosts are not particularly limited as long as the promoter, the target gene, or the environmental stress responsive transcription factor can be expressed therein, and plants are preferable. When a host is a plant, transformed plants (transgenic plants) can be obtained in the following manner.

The “plants” which are to be transformed in the present invention refer to any of a whole plant, plant organs (e.g., leaves, flower petals, stems, roots, or seeds), plant tissues (e.g., epidermis, phloem, parenchyma, xylem, or vascular bundle), or cultured cells of plants. Plants used in the transformation include, but are not limited to, those belonging to Brassicaceae, Gramineae, Solanaceae, or Leguminosae, etc. (see below).

Brassicaceae: Arabidopsis thaliana

Solanaceae: tobacco plant (Nicotiana tabacum)

Gramineae: maize (Zea mays) and rice (Oryza sativa)

Leguminosae: soybean (Glycine max)

The recombinant vector of the present invention can be introduced into plants by conventional transformation methods such as the electroporation method, the Agrobacterium method, the particle gun method, or the PEG method.

When the electroporation method is employed, for example, the gene is introduced into the host using an electroporation apparatus equipped with a pulse controller under conditions of voltage of 500 to 1,600 V, 25 to 1,000 μF, and 20 to 30 msec.

When the particle gun method is employed, after the preparation of slices or after the preparation of protoplast, the whole plant, plant organs, or plant tissues may be used remaining unchanged. Subsequently, a gene introducing device such as PDS-1000/He of Bio-Rad can be used to treat the prepared samples. Although treatment conditions vary depending on types of plants or samples, treatment is generally carried out at a pressure of approximately 1,000 to 1,800 psi at a distance of approximately 5 to 6 cm.

The target gene can be introduced into plants using a plant virus as a vector. An example of plant virus that can be used is the cauliflower mosaic virus. At the outset, a viral genome is inserted into an Escherichia coli-derived vector or another vector to prepare a recombinant. The target gene is then inserted into the viral genome. The thus-modified viral genome is cleaved out of the recombinant with a restriction enzyme and inoculated into a plant host. Thus, the target gene can be introduced into the plant host.

In the method utilizing the Ti plasmid of Agrobacterium, the target gene is introduced into a plant host by utilizing the characteristic that, upon infection of plants by bacteria belonging to the genus Agrobacterium, a part of the plasmid DNA of the bacteria is transferred into the plant genome. Among the bacteria belonging to the genus Agrobacterium, Agrobacterium tumefaciens infects plants and forms tumors referred to as crown galls. Agrobacterium rhizogenes infects plants to generate capillary roots. These are achieved by the transference of a region referred to as a T-DNA (transferred DNA) region on plasmid that is present in bacteria referred to as Ti plasmid or Ri plasmid to the plant at the time of infection and the incorporation into the plant genome.

DNA which is to be incorporated into the plant genome may be inserted into the T-DNA region on the Ti or Ri plasmid. Thus, the target DNA can be incorporated into the plant genome when a plant host is infected by the bacteria belonging to the genus Agrobacterium.

Tumor tissues, shoots, capillary roots, and the like resulting from the transformation can be used as they are for cell culture, tissue culture, or organ culture. Also, with the use of any conventionally known method of culturing plant tissues, plant bodies can be regenerated by the administration of suitably concentrated plant hormones, such as auxin, cytokinin, gibberellin, abscisic acid, ethylene, or brassinolide.

The vector of the present invention can be introduced not only into aforementioned plant hosts but also into, for example, bacteria belonging to the genus Escherichia such as Escherichia coli, the genus Bacillus such as Bacillus subtilis, or the genus Pseudomonas such as Pseudomonas putida, yeasts such as Saccharomyces cerevisiae or Schizosaccharomyces pombe, animal cells such as COS cells or CHO cells, or insect cells such as Sf9 to obtain transformants. When bacteria such as Escherichia coli or yeast are used as hosts, the recombinant vector of the present invention is preferably capable of autonomous replication in the bacteria. At the same time, the vector is preferably comprised of a promoter, a ribosome binding sequence, the target gene, and a transcription termination sequence. A gene that controls a promoter may also be contained therein.

Methods of introducing the recombinant vector into bacteria are not particularly limited as long as DNA is introduced into the bacteria. Examples thereof include a method using calcium ions and the electroporation method.

When yeast cells are used as hosts, for example, Saccharomyces cerevisiae or Schizosaccharomyces pombe are used. Methods of introducing the recombinant vector into yeast cells are not particularly limited as long as DNA is introduced into the yeast. Examples thereof include the electroporation method, the spheroplast method, and the lithium acetate method.

When animal cells are used as hosts, monkey COS-7 cells, Vero, Chinese hamster ovary (CHO) cells, mouse L cells, or the like are used. Examples of methods of introducing the recombinant vector into animal cells include the electroporation method, the calcium phosphate method, and the lipofection method.

When insect cells are used as hosts, Sf9 cells or the like are used. Examples of methods of introducing the recombinant vector into insect cells include the calcium phosphate method, the lipofection method, and the electroporation method.

Whether or not the gene is incorporated into the host can be confirmed by PCR, Southern hybridization, Northern hybridization, or the like. For example, DNA is prepared from the transformant, a DNA-specific primer is designed, and PCR is carried out. PCR is carried out under the same conditions employed for the preparation of the plasmid. Subsequently, the amplification product is subjected to agarose gel electrophoresis, polyacrylamide gel electrophoresis, capillary electrophoresis, or the like and then stained with ethidium bromide, SYBR Green solution, or the like to detect the amplification product as a single band. Thus, the transformation can be confirmed. Alternatively, the amplification product can be detected by PCR using a primer previously labeled with a fluorescent dye, etc. Further, a method may be adopted in which the amplification product is bound to a solid phase such as a microplate to confirm the amplification product by a fluorescent or enzyme reaction.

### 4. Production of Plant

According to the present invention, transformed plant bodies can be reproduced from the above transformed plant cells or the like. A method of reproduction, wherein callus-like transformed cells are transferred to a medium with a different hormone type and concentration and cultured therein to generate adventive embryos to thereby obtain complete plant bodies, is employed. Examples of medium that can be used include LS medium and MS medium.

The method of tissue-specific gene expression of the present invention comprises a step of introducing the expression vector comprising the environmental stress responsive promoter inserted therein into a host cell to obtain transformed plant cells, reproducing a transformed plant body from such transformed plant cells, and obtaining plant seeds from the resulting transformed plant body to produce a plant body of interest from the plant seeds.

Plant seeds are obtained from the transformed plant body by, for example, sampling the transformed plant body from the rooting medium, transferring the plant body into a pot containing hydrated soil, allowing the plant body to grow at constant temperature, produce flowers, and then generate seeds in the end. A plant body is produced from seeds by, for example, isolating seeds when they mature on the transformed plant body, sowing the seeds in water-containing soil, and allowing them to grow at constant temperature and illuminance to produce a plant body. The thus-grown plant allows tissue-specific induction of expression of genes located downstream of the environmental stress responsive promoter of the present invention, in particular.

The correlation between the environmental stress responsive promoter of the present invention and tissues that allows specific induction is shown in Table 3.

TABLE 3

| Promoter | Tissue allowing specific induction |
|---|---|
| Promoter of RAFL05-17-B13 (SEQ ID NO: 1) | Stem and leaf tissue and root tissue |
| Promoter of RAFL05-18-I12 (SEQ ID NO: 2) | Stem and leaf tissue and root tissue |
| Promoter of RAFL05-20-M16 (SEQ ID NO: 3) | Stem and leaf tissue and root tissue |
| Promoter of RAFL06-07-B19 (SEQ ID NO: 4) | Stem and leaf tissue and root tissue |
| Promoter of RAFL06-16-J10 (SEQ ID NO: 5) | Stem and leaf tissue |
| Promoter of RAFL07-08-I12 (SEQ ID NO: 6) | Stem and leaf tissue |
| Promoter of RAFL09-07-M01 (SEQ ID NO: 7) | Stem and leaf tissue and root tissue |
| Promoter of RAFL08-17-O07 (SEQ ID NO: 8) | Root tissue |

The above promoters induce specific expression in different tissues in accordance with the type of environmental stress to be applied. Specifically, the promoters exhibit the properties shown in Table 4.

TABLE 4

| | | Tissue in which specific induction of |
|---|---|---|
| Promoter | Stress type | expression is realized |
| Promoter of RAFL05-17-B13 | Dehydration | Stem and leaf tissue and root tissue |
| | Salt | Stem and leaf tissue |
| | Low temperature | Stem and leaf tissue |
| | ABA | Stem and leaf tissue (weak) |
| Promoter of RAFL05-18-I12 | Dehydration | Stem and leaf tissue and root tissue |
| | Salt | Not measured |
| | Low temperature | Not measured |
| | ABA | Not measured |
| Promoter of RAFL05-20-M16 | Dehydration | Root tissue |
| | Salt | Not measured |
| | Low temperature | Not measured |
| | ABA | Root tissue |
| Promoter of RAFL06-07-B19 | Dehydration | Stem and leaf tissue and root tissue*¹ |
| | Salt | Not measured |
| | Low temperature | Not measured |
| | ABA | Root tissue |
| Promoter of RAFL06-16-J10 | Dehydration | Stem and leaf tissue |
| | Salt | Not measured |
| | Low temperature | Not measured |
| | ABA | Not measured |
| Promoter of RAFL07-08-I12 | Dehydration | Stem and leaf tissue |
| | Salt | Not measured |
| | Low temperature | Not measured |
| | ABA | Not measured |
| Promoter of RAFL09-07-M01 | Dehydration | Not measured |
| | Salt | Stem and leaf tissue and root tissue |
| | Low temperature | Not measured |
| | ABA | Not measured |
| Promoter of RAFL08-17-O07 | Dehydration | Not measured |
| | Salt | Not measured |
| | Low temperature | Not measured |
| | ABA | Root tissue |

*¹Expression is induced in stem and leaf tissue and root tissue when dehydration stress is applied for 5 hours and expression is induced in leaf tissue and root tissue when dehydration stress is applied for 10 hours.

Because of the properties shown in Table 4, a gene of interest can be expressed in a tissue of interest at the desired timing in accordance with a combination of the promoter shown in Table 4 and an environmental stress type to be applied. In the case of a transgenic plant comprising the promoter of RAFL05-17-B13 (SEQ ID NO: 1) and the gene A downstream thereof, for example, the gene A can be expressed in stem and leaf tissue and in root tissue upon application of dehydration stress, and the gene A can be expressed in stem and leaf tissue upon application of salt stress. In such transgenic plants, accordingly, application of dehydration stress and salt stress at different timings enables regulation of the timing of expression of the gene A in stem and leaf tissue and in root tissue and the timing of expression of the gene A in root tissue. Thus, use of the environmental stress responsive promoter of the present invention enables induction of expression of various genes in a tissue-specific manner.

The present invention is hereafter described in greater detail with reference to the examples, although the technical scope of the present invention is not limited thereto.

### Example 1

In this example, the gene expression levels at the time of application of various types of environmental stresses were analyzed using microarrays. Specifically, the microarrays described in Seki et al., “Monitoring the expression profiles of 7000 Arabidopsis genes under drought, cold, and high-salinity stresses using a full-length cDNA microarray,” Plant J. 31: 279-292, 2002, were used, and analysis was carried out in accordance with the method similar to the method described therein.

Various types of environmental stresses were applied to a plant body under the following conditions. Specifically, low-temperature stress application was carried out by introducing a plate containing a plant body into a low-temperature chamber (4° C.). Dehydration stress application was carried out by capturing the plant body with forceps while refraining from damaging the plant body, roughly removing moisture from the plant body on a Kim Towel, placing the plant body on another plate, placing the plate in a clean bench while leaving the lid open, letting it stand for 2 hours, and then closing the lid. Salt stress application was carried out by dispensing a mixture of MS medium (containing 1% sucrose, 0.1% agar, and 1 mM luciferin) with 250 mM NaCl to the roots of the plant bodies (200 μl per plant body) using a Pipetman. ABA stress application was carried out by dispensing a mixture of MS medium (containing 1% sucrose, 0.1% agar, and 1 mM luciferin) with 100 μM ABA to the roots of the plant bodies (200 μl per plant body) using a Pipetman.

As a plant body to which environmental stress is to be applied, Arabidopsis thaliana (columbia ecotype), which had been cultivated on a germination medium containing Murashige and Skoog Salt, 3% sucrose, and 8% Bactoager for 3 weeks, was used. Cultivation conditions were determined so as to maintain the temperature in the chamber at 22° C. with a 16-hour light period and an 8-hour dark period.

Total RNA was extracted from plant bodies after environmental stress application or from plant bodies to which no environmental stress had been applied using the TRIZOL Reagent (Life Technologies), and mRNA was extracted using the mRNA isolation kit (Militenyi Biotec Auburn). In accordance with the method described in the above article, mRNA samples were reversely transcribed in the presence of Cy3 dUTP or Cy5 dUTP (Amersham Pharmacia). Microarray analysis was carried out using cDNAs obtained via reverse transcription. Data obtained via microarray analysis were analyzed using the Quantarray Version 2.0 (GSI Lumonics).

As a result, genes exhibiting environmental stress responsive expression patterns were identified, as shown in Table 5.

TABLE 5

| | 1 hour | | 2 hours | | 5 hours | | 10 hours | | 24 hours | |
|---|---|---|---|---|---|---|---|---|---|---|
| Gene | Mean | S.D. | Mean | S.D. | Mean | S.D. | Mean | S.D. | Mean | S.D. |
| | Expression ratio (ABA/no treatment) | | | | | | | | | |
| RAFL05-17-B13 (At1g01470) | 5.8 | 1.7 | 7.2 | 1.4 | 4.0 | 1.7 | 5.1 | 0.4 | 2.7 | 0.3 |
| RAFL05-18-I12 (At2g47770) | 4.0 | 0.4 | 8.7 | 2.0 | 10.1 | 2.8 | 14.4 | 3.0 | 23.2 | 6.3 |
| RAFL05-20-M16 (At2g46680) | 6.8 | 2.9 | 7.5 | 2.8 | 8.8 | 1.4 | 18.8 | 2.9 | 13.9 | 9.0 |
| RAFL06-07-B19 (At3g11410) | 5.7 | 1.0 | 8.7 | 2.5 | 7.6 | 2.3 | 16.0 | 2.8 | 12.0 | 5.0 |
| RAFL06-16-J10 (At2g06050) | 7.8 | 3.5 | 3.7 | 0.6 | 1.4 | 0.9 | 1.8 | 0.3 | 1.2 | 0.4 |
| RAFL07-08-I12 (At2g26530) | Undetectable | Undetectable | 3.0 | 1.3 | 1.5 | 0.8 | 1.2 | 1.2 | 2.1 | 1.6 |
| RAFL09-07-M01 (At4g20830) | 4.5 | 2.5 | 3.1 | 2.2 | 1.8 | 0.6 | 5.0 | 0.2 | 3.0 | 1.1 |
| RAFL08-17-O07 (At2g29450) | 12.9 | 6.7 | 8.6 | 5.0 | 3.0 | 1.9 | 12.0 | 2.3 | 11.3 | 7.3 |
| | Expression ratio (low temperature/no treatment) | | | | | | | | | |
| RAFL05-17-B13 (At1g01470) | 1.6 | 0.0 | 2.1 | 0.2 | 4.5 | 0.7 | 10.2 | 4.7 | 5.6 | 0.9 |
| RAFL05-18-I12 (At2g47770) | 1.9 | 0.6 | 2.0 | 0.5 | 1.5 | 0.5 | 1.6 | 0.3 | 1.4 | 0.2 |
| RAFL05-20-M16 (At2g46680) | 1.1 | 0.0 | 2.0 | 0.1 | 1.6 | 0.2 | 1.4 | 0.4 | 1.3 | 0.1 |
| RAFL06-07-B19 (At3g11410) | 1.3 | 0.2 | 3.2 | 0.4 | 1.2 | 0.3 | 1.3 | 0.3 | 1.0 | 0.3 |
| RAFL06-16-J10 (At2g06050) | 2.2 | 0.4 | 2.5 | 0.2 | 1.2 | 0.2 | 1.0 | 0.3 | 0.8 | 0.3 |
| RAFL07-08-I12 (At2g26530) | 1.4 | 0.3 | 3.6 | 0.4 | 1.1 | 0.7 | 1.5 | 0.2 | 0.8 | 0.3 |
| RAFL09-07-M01 (At4g20830) | 1.4 | 0.4 | 2.2 | 1.6 | 2.1 | 1.0 | 2.6 | 1.0 | 2.7 | 1.3 |
| RAFL08-17-O07 (At2g29450) | 1.3 | 0.3 | 0.9 | 0.6 | 1.0 | 0.3 | 1.7 | 0.7 | 0.6 | 0.1 |
| | Expression ratio (dehydration/no treatment) | | | | | | | | | |
| RAFL05-17-B13 (At1g01470) | 3.3 | 0.2 | 12.3 | 3.2 | 13.8 | 5.2 | 13.8 | 2.1 | 10.6 | 3.9 |
| RAFL05-18-I12 (At2g47770) | 3.2 | 2.2 | 26.7 | 12.5 | 39.8 | 25.7 | 73.4 | 36.5 | 64.9 | 64.4 |
| RAFL05-20-M16 (At2g46680) | 1.3 | 0.1 | 3.3 | 1.4 | 3.0 | 1.0 | 9.2 | 1.3 | 9.3 | 4.0 |
| RAFL06-07-B19 (At3g11410) | 1.5 | 0.6 | 3.7 | 2.5 | 2.3 | 0.6 | 4.5 | 0.3 | 5.4 | 2.9 |
| RAFL06-16-J10 (At2g06050) | 4.8 | 1.1 | 6.4 | 1.9 | 1.5 | 0.5 | 1.1 | 0.2 | 0.8 | 0.2 |
| RAFL07-08-I12 (At2g26530) | 1.9 | 0.8 | 2.9 | 1.8 | Undetectable | Undetectable | 1.2 | 0.1 | 1.2 | 0.2 |
| RAFL09-07-M01 (At4g20830) | 5.3 | 3.8 | 4.3 | 2.8 | 2.3 | 1.0 | 3.8 | 1.5 | 4.6 | 3.3 |
| RAFL08-17-O07 (At2g29450) | 1.9 | 0.9 | 4.6 | 3.3 | 2.6 | 0.8 | 4.0 | 1.4 | 2.6 | 1.1 |
| | Expression ratio (salt stress/no treatment) | | | | | | | | | |
| RAFL05-17-B13 (At1g01470) | 5.7 | 1.7 | 6.7 | 1.3 | 5.7 | 0.8 | 4.4 | 1.9 | 4.1 | 3.2 |
| RAFL05-18-I12 (At2g47770) | 3.5 | 0.9 | 11.2 | 6.8 | 33.8 | 23.5 | 14.7 | 11.4 | 38.7 | 32.0 |
| RAFL05-20-M16 (At2g46680) | 3.6 | 0.8 | 4.9 | 1.6 | 5.6 | 3.3 | 4.9 | 0.2 | 3.3 | 2.5 |
| RAFL06-07-B19 (At3g11410) | 3.2 | 0.6 | 2.9 | 1.6 | 3.8 | 1.6 | 3.2 | 1.6 | 3.4 | 2.3 |
| RAFL06-16-J10 (At2g06050) | 5.8 | 2.4 | 3.0 | 0.5 | 2.1 | 0.3 | 1.6 | 0.5 | 1.4 | 0.6 |
| RAFL07-08-I12 (At2g26530) | 3.2 | 1.3 | 2.6 | 0.2 | 2.2 | 0.3 | 1.2 | 0.2 | 1.9 | 1.0 |
| RAFL09-07-M01 (At4g20830) | 7.1 | 3.8 | 4.4 | 1.2 | 3.5 | 0.3 | 2.6 | 0.8 | 2.8 | 0.9 |
| RAFL08-17-O07 (At2g29450) | 9.4 | 3.7 | 6.1 | 1.0 | 5.9 | 2.1 | 4.0 | 0.6 | 3.7 | 1.8 |

### Example 2

In Example 2, the promoter activity of the genes exhibiting environmental stress responsive expression patterns identified in Example 1 was examined. Specifically, promoter regions were isolated from these genes, transgenic plants in which luciferase reporter genes were to be expressed under the control of such promoters were prepared, and tissue specificity of the promoters was inspected via luciferase assay.

Protocol of preparation of recombinant vector and transgenic plant:

### (1) Preparation of Recombinant Vector

In this example, DNA fragments containing promoter regions of the genes identified in Example 1 were recovered via PCR. Primer sets shown in Table 6 were used for PCR.

TABLE 6

| | | |
|---|---|---|
| Gene | forward primer | reverse primer |

| | | |
|---|---|---|
| RAFL05-17-B13 | GGGGACAAGTTTGTACAAAAAAGCAGGC | GGGGACCACTTTGTACAAGAAAGCTGGGT |
| (At1g01470) | TCCGCCAACTACCAACACCG | AATAACTCTTCTTGTTTAAATCTC |
| | (SEQ ID NO: 9) | (SEQ ID NO: 10) |
| RAFL05-18-I12 | GGGGACAAGTTTGTACAAAAAAGCAGGC | GGGGACCACTTTGTACAAGAAAGCTGGGT |
| (At2g47770) | TACCATGGCACCTGAAATACG | TACAAACGTCCAAAACAGAATCG |
| | (SEQ ID NO: 11) | (SEQ ID NO: 12) |
| RAFL05-20-M16 | GGGGACAAGTTTGTACAAAAAAGCAGGC | GGGGACCACTTTGTACAAGAAAGCTGGGT |
| (At2g46680) | TGGGAATCTGCCTCAAATATGG | CTCATCGGAATTTTTCCTCAGAGG |
| | (SEQ ID NO: 13) | (SEQ ID NO: 14) |
| RAFL06-07-B19 | GGGGACAAGTTTGTACAAAAAAGCAGGC | GGGGACCACTTTGTACAAGAAAGCTGGGT |
| (At3g11410) | TCCCGAACTTAACCCAAATGCCC | TTGATCTCTAACAAAACTTCTCC |
| | (SEQ ID NO: 15) | (SEQ ID NO: 16) |
| RAFL06-16-J10 | GGGGACAAGTTTGTACAAAAAAGCAGGC | GGGGACCACTTTGTACAAGAAAGCTGGGT |
| (At2g06050) | TCCTGGGACTTGGGCTGAG | GTCTCCGCCGATCTGGAAG |
| | (SEQ ID NO: 17) | (SEQ ID NO: 18) |
| RAFL07-08-I12 | GGGGACAAGTTTGTACAAAAAAGCAGGC | GGGGACCACTTTGTACAAGAAAGCTGGGT |
| (At2g26530) | TCGGGTTTTATTTTGGAATTGG | TGTTTCTAGTTTCCTTTGAGTTCGG |
| | (SEQ ID NO: 19) | (SEQ ID NO: 20) |
| RAFL09-07-M01 | GGGGACAAGTTTGTACAAAAAAGCAGGC | GGGGACCACTTTGTACAAGAAAGCTGGGT |
| (At4g20830) | TAGGTGCAGGAGATTGAATCG | TTTGAGATCTTTTTCTTGGGTCTCG |
| | (SEQ ID NO: 21) | (SEQ ID NO: 22) |
| RAFL08-17-O07 | GGGGACAAGTTTGTACAAAAAAGCAGGC | GGGGACCACTTTGTACAAGAAAGCTGGGT |
| (At2g29450) | TCGGTGGCAAAACAATTGG | TAGGGGTCTCTCTCTCTTTTTCTC |
| | (SEQ ID NO: 23) | (SEQ ID NO: 24) |

The nucleotide sequences of the amplified DNA fragments were determined in accordance with a conventional technique to confirm that mutagenesis, such as substitution or deletion, was not introduced thereinto. After the absence of mutagenesis was confirmed, the recovered DNA fragments were introduced into the vector for promoter analysis (i.e., a vector prepared by introducing the Gateway recombinant sequence (tradename: Gateway Vector Conversion System, Invitrogen) and the luciferase reporter gene into the pGreen vector (Plant Molecular Biology 42: 819-832, 2000)) using the Gateway recombination system to construct a recombinant vector.

### (2) Preparation of Transgenic Plant

The recombinant vector prepared in (1) above was introduced into a plant via Agrobacterium infection. When a gene is introduced via Agrobacterium infection, a process of infecting a plant with Agrobacterium containing a plasmid comprising the target gene construct is necessary. This process was carried out via vacuum infiltration. Specifically, a plant body of Arabidopsis thaliana that had been grown in soil comprising vermiculite and an equivalent amount of pearlite was soaked in a culture solution of Agrobacterium containing the recombinant vector prepared in (1). The resultant was placed in a desiccator, suctioned with a vacuum pump to 65 to 70 mmHg, and then allowed to stand at room temperature for 5 to 10 minutes. The pot was transferred onto a tray, covered with a wrap, and kept moistened. The wrap was removed on the following day, the plant was allowed to grow in that state, and seeds were then harvested.

Subsequently, seeds were sown on MS agar medium to which antibiotic hygromycin has been added in order to select an individual having the recombinant vector prepared in (1). Arabidopsis thaliana that had been grown in this medium was transferred to a pot and allowed to grow therein. Thus, seeds of transgenic plants to which the recombinant vector prepared in (1) has been introduced were obtained.

### (3) Protocol of Luciferase Assay

The seeds of the transgenic plant lineage prepared in (2) were sowed on MS agar medium. The plant body 10 days after sowing was used for luciferase assay.

At the outset, 1 mM luciferin (containing 0.01% Triton-X) was sprayed over the entire plant body 5 times. After the plant body was allowed to stand in the dark for 5 minutes, luciferase luminescence was assayed using ARGUS (assay after treatment for 0 hours). Subsequently, various types of environmental stresses were applied (see below). Low-temperature stress application was carried out by introducing a plate containing a plant body into a low-temperature chamber (4° C.). Dehydration stress application was carried out by capturing the plant body with forceps while refraining from damaging the plant body, roughly removing moisture from the plant body on a Kim Towel, placing the plant body on another plate, placing the plate in a clean bench while leaving the lid open, letting it stand for 2 hours, and then closing the lid. Salt stress application was carried out by dispensing a mixture of MS medium (containing 1% sucrose, 0.1% agar, and 1 mM luciferin) with 250 mM NaCl to the roots of the plant bodies (200 μl per plant body) using a Pipetman. ABA stress application was carried out by dispensing a mixture of MS medium (containing 1% sucrose, 0.1% agar, and 1 mM luciferin) with 100 μM ABA to the roots of the plant bodies (200 μl per plant body) using a Pipetman.

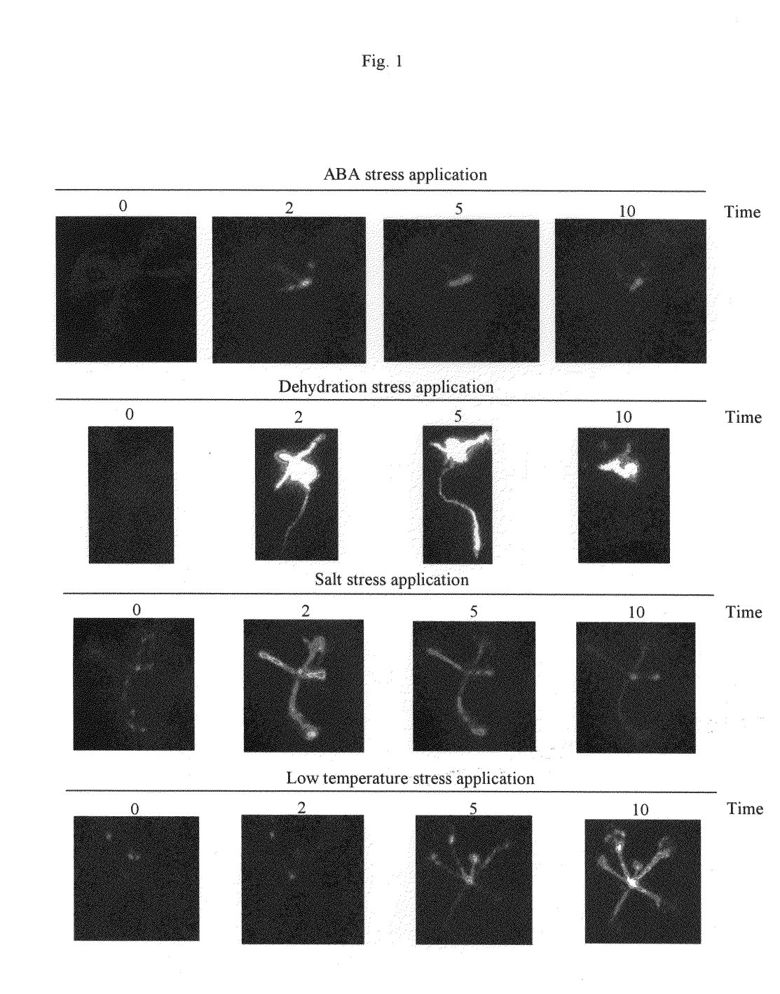

Luciferase luminescence was assayed 2, 5, and 10 hours after the above various types of environmental stresses had been applied using the ARGUS system (Hamamatsu Photonics). Before assay, 1 mM luciferin (containing 0.01% Triton-X) was sprayed over the entire plant body 5 times, and the plant body was allowed to stand in the dark for 5 minutes. The results are shown in FIGS. 1 to 8.

As shown in FIG. 1, the promoter of RAFL05-17-B13 was found to induce gene expression in stem and leaf tissue and root tissue upon dehydration stress application. Also, the promoter of RAFL05-17-B13 was found to induce gene expression in stem and leaf tissue upon salt stress or low temperature stress application. Further, the promoter of RAFL05-17-B13 was found to induce weak gene expression in stem and leaf tissue upon ABA stress application. Thus, the promoter of RAFL05-17-B13 exhibited interesting features such that the promoter would induce gene expression in different tissues in accordance with the type of environmental stress applied.

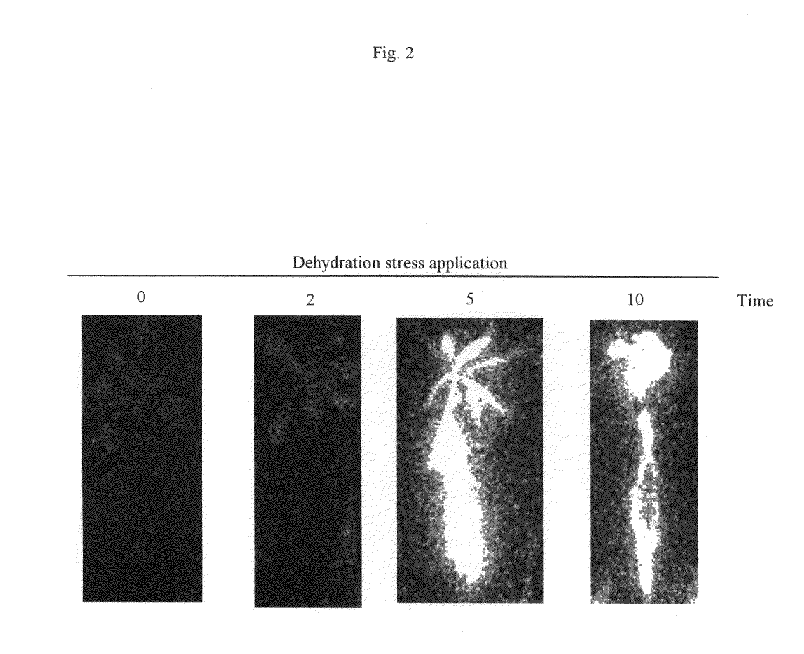

As shown in FIG. 2, the promoter of RAFL05-18-I12 was found to induce gene expression in stem and leaf tissue and root tissue upon dehydration stress application. Also, the promoter of RAFL05-18-I12 exhibited interesting features such that it would exhibit strong activity to induce gene expression 5 hours after dehydration stress application.

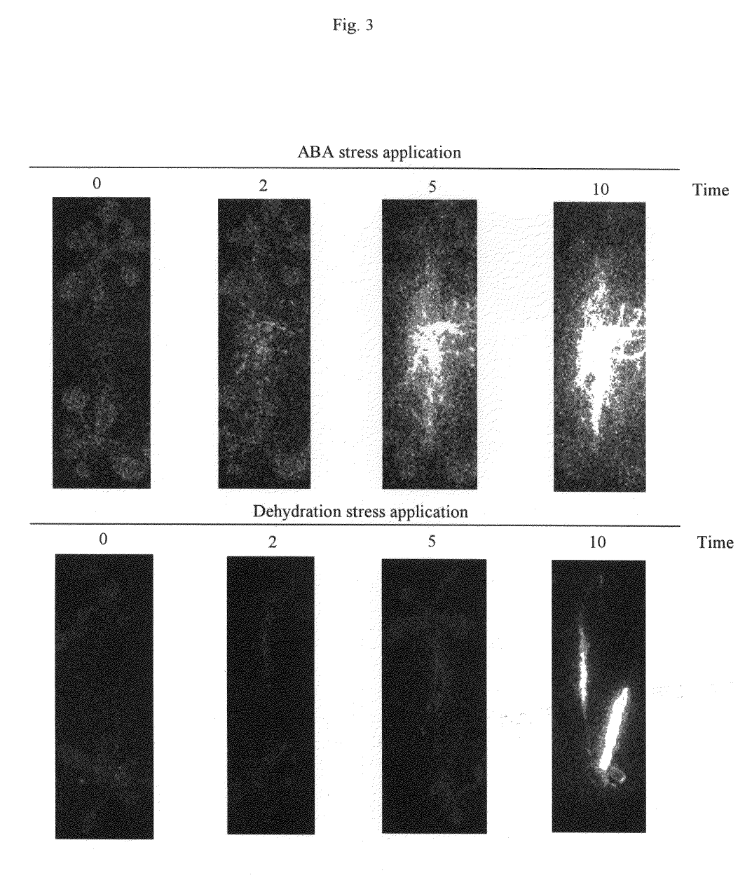

As shown in FIG. 3, the promoter of RAFL05-20-M16 was found to induce gene expression in root tissue upon dehydration stress or ABA stress application. Also, the promoter of RAFL05-20-M16 was found to exhibit activity to induce gene expression in root tissue about 10 hours after dehydration stress application and to exhibit such activity in root tissue about 2 hours after ABA stress application.

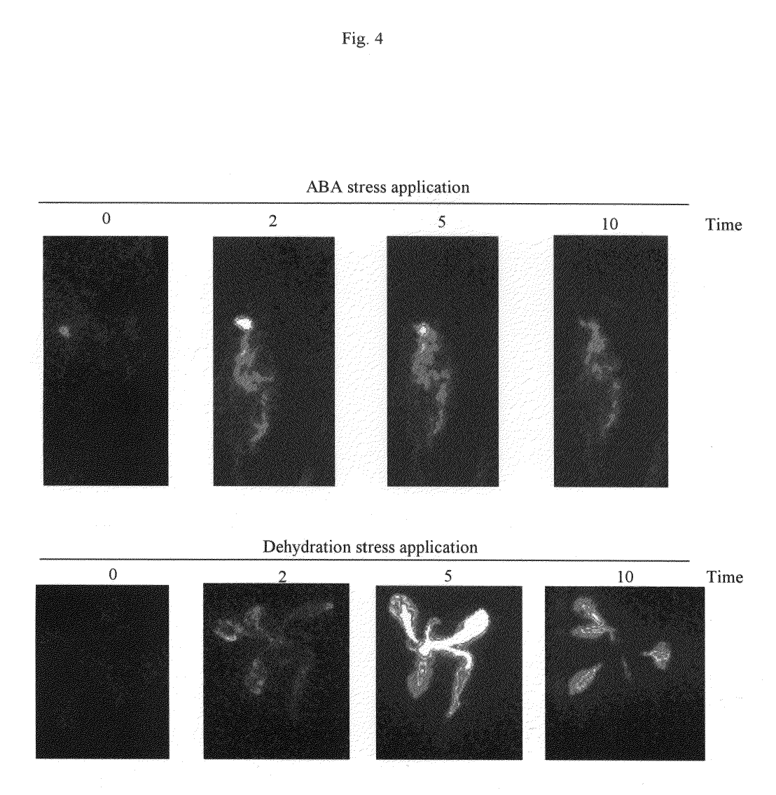

As shown in FIG. 4, the promoter of RAFL06-07-B19 was found to induce gene expression in stem and leaf tissue and root tissue upon dehydration stress application. Also, the promoter of RAFL06-07-B19 was found to induce gene expression in root tissue upon ABA stress application. Further, the promoter of RAFL06-07-B19 was found to induce gene expression in stem and leaf tissue and root tissue about 5 hours after dehydration stress application; however, it was found to exhibit lowered activity to induce gene expression in stem tissue and root tissue but to induce gene expression in leaf tissue about 10 hours after stress application.

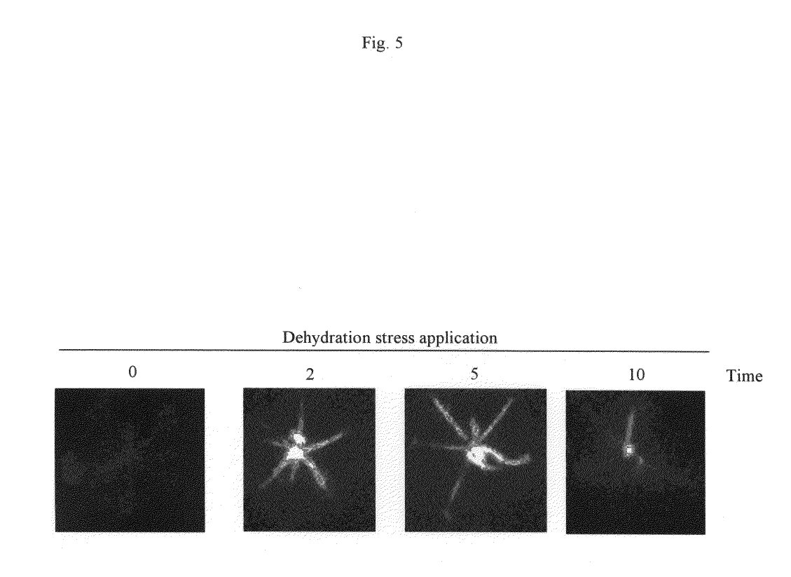

As shown in FIG. 5, the promoter of RAFL06-16-J10 was found to induce gene expression in stem and leaf tissue upon dehydration stress application. Also, the promoter of RAFL06-16-J10 was found to exhibit activity to induce gene expression in stem and leaf tissue about 2 hours after dehydration stress application but to exhibit lowered activity about 10 hours after stress application.

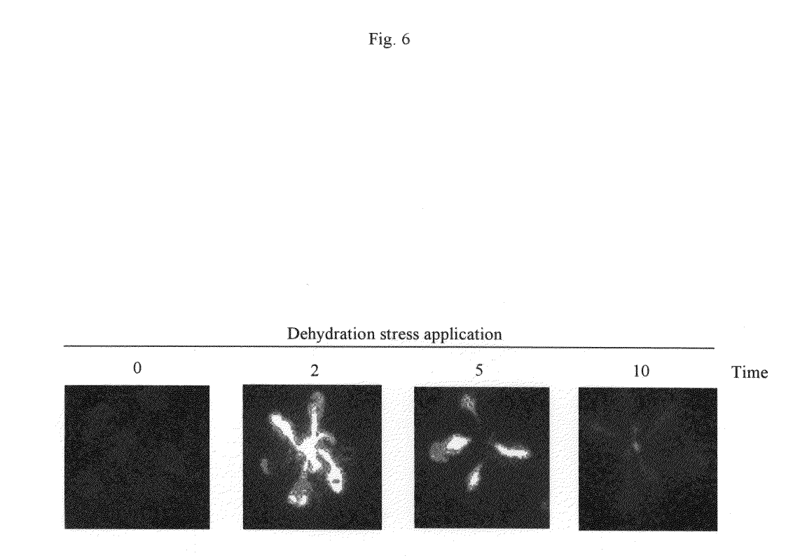

As shown in FIG. 6, the promoter of RAFL07-08-I12 was found to induce gene expression in stem and leaf tissue upon dehydration stress application. Also, the promoter of RAFL07-08-I12 was found to exhibit activity to induce gene expression in stem and leaf tissue about 2 hours after dehydration stress application but to exhibit lowered activity about 5 hours after stress application.

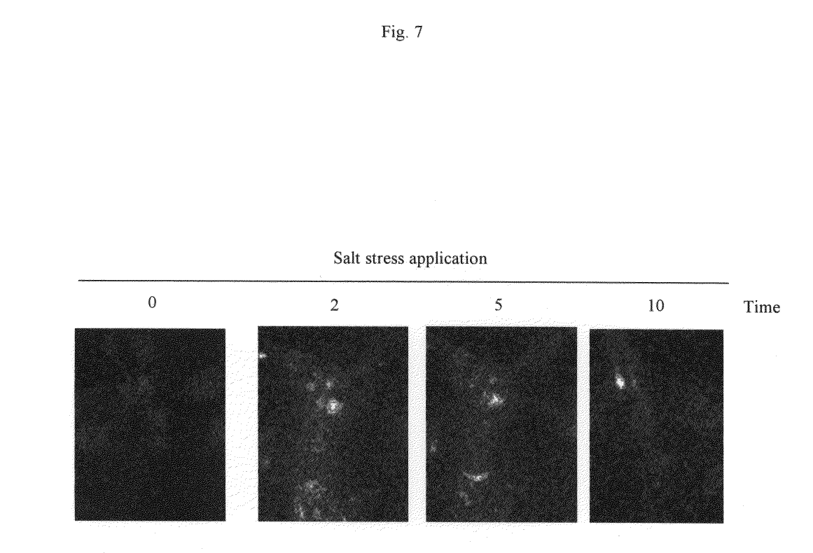

As shown in FIG. 7, the promoter of RAFL09-07-M01 was found to induce gene expression in stem and leaf tissue and root tissue upon salt stress application. Also, the promoter of RAFL09-07-M01 exhibited features such that activity to induce gene expression thereof was lower than that of other promoters.

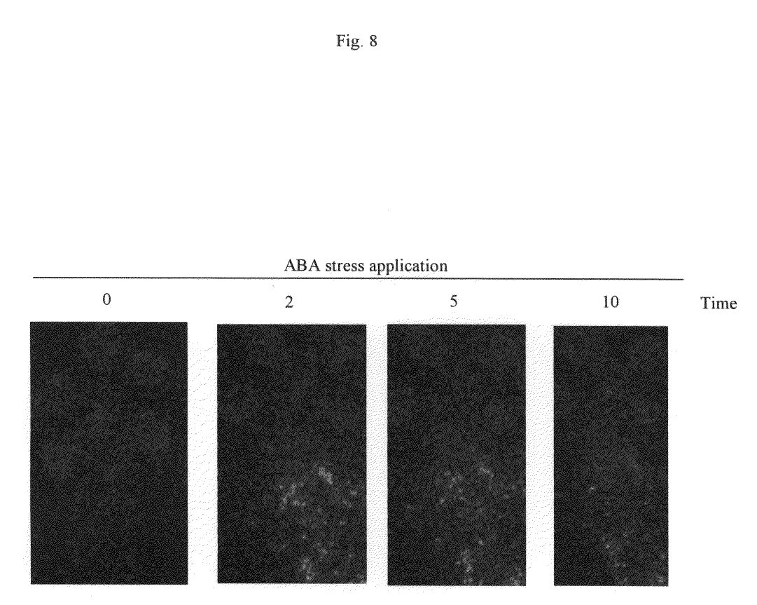

As shown in FIG. 8, the promoter of RAFL08-17-O07 was found to induce gene expression in root tissue upon ABA stress application. Also, the promoter of RAFL08-17-O07 exhibited features such that activity to induce gene expression thereof was lower than that of other promoters.

As shown in FIGS. 1 to 8, promoters of the environmental stress responsive genes identified in Example 1 exhibited characteristic activities to induce gene expression. Patterns of activities to induce expression of the promoters obtained in this example may be adequately used to express the target genes at a timing, in tissue, and at a level desired. Thus, promoters of the environmental stress responsive genes identified in Example 1 were found to be useful when constructing an experimentation system that can regulate gene expression in a plant body in a tissue-specific and/or time-specific manner.

### INDUSTRIAL APPLICABILITY

The present invention can provide a novel promoter that has responsiveness to various types of environmental stresses and that is capable of inducing gene expression specifically in given tissue. Use of the promoter of the present invention enables expression of a desired gene specifically in tissue, such as stem and leaf tissue or root tissue. The present invention, accordingly, can be used for molecular breeding with desired properties, such as crops exhibiting stronger tolerance to environmental stress.

All publications, patents, and patent applications cited herein are incorporated herein by reference in their entirety.

1. An environmental stress responsive promoter comprising DNA (a), (b), or (c) below:
(a) DNA comprising the nucleotide sequence as shown in any of SEQ ID NO: 1 and 4 to 8;
(b) DNA comprising a nucleotide sequence derived from the nucleotide sequence as shown in any of SEQ ID NO: 1 and 4 to 8 by deletion, substitution, or addition of one or a plurality of nucleotides and functioning as an environmental stress responsive promoter; or
(c) DNA hybridizing under stringent conditions to DNA comprising the nucleotide sequence as shown in any of SEQ ID NO: 1 and 4 to 8 and functioning as an environmental stress responsive promoter.

2. The promoter according to claim 1, wherein the environmental stress is at least one stress selected from the group consisting of low temperature stress, dehydration stress, and salt stress.

3. The promoter according to claim 1, which functions in stem and leaf tissue and/or root tissue.

4. An expression vector comprising the promoter according to claim 1.

5. An expression vector further comprising an arbitrary gene incorporated into the expression vector according to claim 4.

6. A transformant comprising the expression vector according to claim 4 or 5.

7. A transgenic plant comprising the expression vector according to claim 4 or 5.

8. The transgenic plant according to claim 7, wherein the plant is a plant body, a plant organ, plant tissue, or cultured plant cells.

9. A method for producing a stress tolerant plant comprising culturing or cultivating the transgenic plant according to claim 7.

10. A method of tissue-specific gene expression comprising:
a step of preparing a plant having an arbitrary gene downstream of the environmental stress responsive promoter comprising DNA (a), (b), or (c) below;
(a) DNA comprising the nucleotide sequence as shown in any of SEQ ID NO: 1 to 8;
(b) DNA comprising a nucleotide sequence derived from the nucleotide sequence as shown in any of SEQ ID NO: 1 to 8 by deletion, substitution, or addition of one or a plurality of nucleotides and functioning as an environmental stress responsive promoter; or
(c) DNA hybridizing under stringent conditions to DNA comprising the nucleotide sequence as shown in any of SEQ ID NO: 1 to 8 and functioning as an environmental stress responsive promoter; and
a step of cultivating the plant in the presence of environmental stress,
the method comprising inducing expression of the gene located downstream of the environmental stress responsive promoter in a tissue-specific manner.

11. The method of tissue-specific gene expression according to claim 10, wherein the environmental stress is at least one stress selected from the group consisting of low temperature stress, dehydration stress, and salt stress.

12. The method of tissue-specific gene expression according to claim 10, wherein the gene is induced to express specifically in stem and leaf tissue and/or root tissue.

13. The method of tissue-specific gene expression according to claim 10, which further comprises a step of introducing an expression cassette comprising the above gene located downstream of the environmental stress responsive promoter into the plant.


SEQUENCE LISTING (24 sequences)

SEQ ID NO: 1 - DNA, 1338 bp, Arabidopsis thaliana
ccgccaactaccaacaccgacatatgtgctttcggcagttttgatatcattccaaaatttcaattattatttatcaaatttattttcttggtaaaagtttaattaatttcaattatttgaatgtcttaattcactgattacaaattatatatgtaatatatacataacaatttcgttcgtggagtgttattatactccttaagcgaatctgtatattttaaataaaattttgatttatgcacacgtacatgttcaattgaaacgttttccctaactatagatgccaataaataataagttgatttttaaaatgtacaaaacaaaaggtaccgttttgtaaaattttgcaaccaaaaagtacaatcggccgttgaaaatcgtggaaccaaacactaattaaagagaagtgtgaatataatctagtgtttgcggatttatattataaaatttgtgtatcactaaaatgtcaaagcatgctttgactggtcaataagtcaaattaactaacaaaccatgcgaggcgtaggtttgacttcttaacataaatgctgactcagtttttccattccgcagctgtcgactaattggacacgcttcttccaatccctttccactaatcatccatcaacacgtgacatgcctacagtgcacgacttcacagtgatcatgtcaaagttttgtttttaagagaagaagattcacgacttcacagtgatcatagtccagttacaaatttgaatgggcttatggcctgtggcccatttaagtttgaccttaatatccatcagaaacccataccaatacgggtcataagtgcattgtccatatagtgtaccgcaatgcatgggagtactactacattccaccgacgtgcatatggggggacatggtcggtccattaatcccttgcgcatcttattccttacgcgaatacttttccctcttttagttttgtataatactttccatttttttggtattaaatttatcgtgtgactatctaaaaaggtatttaattatctagtttagctataactgcaaacaaaatatctattttcatgaatgatatatttttgaaataaaatattgttgtcatgtaacgaaaaaatcaaaaatcaagcagaagacacgtataacccacgctttcactctcactcgtggaaaaccctcacacgtacacaaaccattataaaatttaatccatttacatcgaccgacttcataagaacatcgtaatcccatccgtttatatatatattccaagttaaacttcatatcatacacacaaacctaaaacaccgaaacaaaaacaaagagatttaaacaagaagagttatt

SEQ ID NO: 2 - DNA, 1356 bp, Arabidopsis thaliana
accatggcacctgaaatacgtaaggatacaagaagagatatcatcactgaattcactaaaatcactcccatcataaacagacaaatcaaaaccaaaatattgttcattaacatcgtaaatggacaacgcttgacaacgtctacaaatacttgatgaaaagagataagaaataagtcttcatttataccacatcagtcttcacgtagatgatcaagacgatccccagattgacaatgtagagaacaccaaaaagaatctgcaaaatgtaagagagtagaattgagacaaatgaagcgatggatggatcaaattccgcagctaatccaaagataggagattcaaagagagggaattgcaagcacctgagcagggtagacttcccctccagttaagttctgcactgcagagtaaacatagaggaaacctgctggataaaccaatggccctgtatcgcctttcaagttcccatagtctctctctcccccgagaaacccactaacctgccccaattagataaatttcacttaaagactactagtttactttgaagaaaccacagttgaccattgacacttacctgtgacatgtaagcatcccaatcgatcttcgtgtctgttaaaggcaaatgacgaaatttggtttaaaacctatgcaaatgcacatttcgagggatacgcaagcaaaaaaaaaaaggaggtaaatagctctcacatggaacataggcgataataagtgcgactaggattgcatcagcaagaattagcgcgaatgcgaatggaactgcaggttttttgaatagatcggatcgattcgtctccttccccagccgacggctacgagaagctctcaaactcgccggtgatgaggcgcccgccatgaaaacagagcaaatcgcatcagcgtctagccaacgccgcgtaacagacaactacttccatattactactcttctaattagcccaaattaaatgagcctattgggcttcttgtcttagtcggtgtagagcccaattgttgttttattttttaataatgcaaaagtattaagcgataaataaataagcatcgcaatcgtcccaaaactgtgtgtatgcatcagacatgagcatatagagtaagcacgtgtccacactttttcacaaagttatctaaaaacaaaaaacaattaattagcattcgacgtgtacatatcactcgccacgtgtacaagagccttggcctttttgcttcttcttcttgtctattaatatcatctcctgattatactctcttttgaccaagctgcttcttctccatctatccaacatctcaaatctcagtaatcttgttccttcgattctgttttggacgtttgta

SEQ ID NO: 3 - DNA, 1614 bp, Arabidopsis thaliana
gggaatctgcctcaaatatggtttccctgttggcaaaaatttaacttgcttccattcaaatgatgaaatgttacgaaaaattagccttttgtctttggagtaatgtttgttaaataatcacgaggcattcattcttaaaataccataacatttatagaggtttgaagtaccataatttgtaaaactaaactagagaaattttgcgaaatcagttgtatgaagactttcttaaaaataacgttatatagactctagaaaaaaactattatatgccatttctatttagtttttttttctaaaaatatatataaatatggtgttgttaacataaaaacagaaaaaaagaaaaatttagaaaattttctcaagaatcattatatctgtgatttatcatataagttcaaatatgatattagaaacaaatagtttacgagtataatagtattgtcaatttttcaatctgaagtaaatatctttttgtaaggaaggggtcaacaaatgatcacaacagagttggcaaaaagttatcaaatcgcatgcacggaagttttacgtgtggtgaaggtaaacttgtattacacttatctataaaaattagtttaggctttgattctaaatcaaatctccgattagaaaaaattgcgtaagcaaatagctggaaaaaattgtatcccatcatacttaagtcacaatgttttgtttttgagatttgtgatgtaatcaatatatgttttacaatgcaagtataataatattaaagtcacattctaagaaaattatgatttgtgtcatacgtatacaaaaacacccgtcacacatcctgacttctgaacgttaaatctgtcgcacacaatcataaaaatttaaaaattcaccagagatgtactgaaaagaatataattaatcacatgatgatatatgcataggagatgaggattattcattttctgaaattccctatatgaaccattataattgtttagtaatcagttcagaaatgctaatcattatatgaaccattataattcccttcatttttatttaagatccacttaacaggatttgttaatatgcacccacatcactaaatacattggtacgcaaccgttgttccatttccattttcacatcgaccagaatgtttactatgcggtaaattgtgtagtatgcagatttttttgtatcatttaattttctaacacttgttaagtcgaaactaattttgtcacaagtaaaagaaataaaaaaggtggaaattattaatcagtagttagatgattagtttcgagttgaaatgaaactcgacttaacaagtgatagcgacgactctagaaacagccaaaatccgccctattgctacctgtcgacccacaaatcgtttactcaaaaatgaataaaaaatttacgataaagcaaacccaaagttatatcttattatatgctaaggagccctcccaaaaaagaacaacaaatcacatttttatataactgttaacataataatctcagcctcatcaacacacatatatagatagccaacatcacacaaacatagagattccaaaaaataaaaataaagaaaacataaatcctctgaggaaaaattccgatgag

SEQ ID NO: 4 - DNA, 1475 bp, Arabidopsis thaliana
cccgaacttaacccaaatgcccacctttaattattcaaaaccaaaaaagaagtgtttgatgctgaggtttgaactcatcagatacacgtaactattttccaatttattttcgtttatgtgcatttcagaatttcaagcataaatatactcttattgtttaaacatttcaaattgcatgtgtgaaataatatcagagttagaatatatgttaatcttctcttctcctagattcagttagatacgtttctatcttcttttaggcaacatctcttgtacaaatagtctctttggcaattcaatacaatcaaccttttacgatatatatatttgttatttcttgtacaaataggatcaaaattgttatttctgaagcaaattttctttagtttggatgtcaattatatcctcgattaatatgtaatcacggaacggattcacgatgcgttattttagaagcaaatttatacttgatagtacgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 5 - DNA, 1478 bp, Arabidopsis thaliana
cctgggacttgggctgagaataatgaagtggctgggaaatgattagttaaggatggcttgatgaattctcaatattctgttgagggaacaaaatatactaggaaggatgcaagtccagtttggtctaaaccattgtacgaaccaaaaacaatcaataaagtaactcagggctcacaaatggtcgataatatttctaaagtttcagttctagaggatgagcaaaaggatttgatgatgggagatgtactagaggtacaagacaagagtctcactgaagggttgaatttcagatattttaaatcacgtttggagtaacaaaaaacaaatgtgagaaggagctcaattagctccaactgaatctagtgttattagggatactttggaaactccaattcaatctttaagatgtaggattgatgaattcatggaaatcggcagatccatctatgaatttagtatggtggtgtttcctaggaattgacaatacaattttacttggtgcaaagtgtcttcggaggtgtccctttctttttcactcggaattggaagttttaatattggcaataaaaaatatcctttcaagaaggataaagtgtcaggcatttgaaacagataattattcagagtaagttttgatggtacagtcccctgaagaatgactcgctttctctaattttttagaagatcttgaattgctatgtgtctccttttcttgctttcttctatttcatgtttatgtaatactaaggcaaactatttagcacaatattctaaacttttattttctgaagttattttttaaaccctttccctcctaattggacaaccaatcatggaattagtttttacgtaatgttttaaactcactatttcacctttattctgacctgttaatggaagacaactatgactttatacattgaaatgttgtcaacctgttatatattaatgttaacatagctaatgacctatcaatgtacgtaaagcgaatgtcaatcatattcgtatccgttaatttcatttaggaaatcaggctcattgccaatccactaagaaacatctctgatagatccaaataataaaaataatataatatgaagcaaaatagaacgaaatatctcacgcggaccaaactttgaactttattataaaacaaatcagcacgtgaaagtaatggtcggttcggtccaagatgccaagaactcctccggtccatccatcaacctaaccttttttggatataaaatttagcaaattccatttccacgaccacacaacaacaacacatccactcgaattttctatttccgaataacaaaaccggtaaactcatcacaaacttaagttactttgtagtttgtgttttgtccttttatgtaatgaagaagatccaactaactactactcaatatttttgtattgaccgttgatttctcagatctgacttttacttctccttcttccagatcggcggagac

SEQ ID NO: 6 - DNA, 1344 bp, Arabidopsis thaliana
cgggttttattttggaattgggttatatggtggatgtatttgaatcaatatttataaaaatttaacaattatctcatggattcgtggtataacgttacctaataacaattataaactgtaaaataaaaatattttataaaaataaaatttgcaagttttaatatatattacttttaaaaaataaatcgtcccgcggtataccgcgggttaaaatctagtctatgtttattataaaagaatacaatgtattaatgtataaattatcttatgcatgagcacgatgatcatctagtagtatacatcttacccgtcgacaaaaacaaaatgttccttatccagtatctagatggatcgtgtgaactcaattctacgtagaacacaaaagtcctctcgtttggtcactgaattaatttgaacgatattatttattaggaccaccggttaaattggcctcgtggtctaaatatgccttcgaatggtccaacaaccatgtggtcaatgccgactatgaaaatgactgaaaattcaaaacatgtaagtgggttaccaccacttatcattatgttccaaatcgtttgcattaaacaactttatttcgacacatgcggtctgaaaaggcggccaattaatttacgttaatcatcgtttactattattatcttcggaatatttccaacacaatcaatctagttggttccaattctcatcacatattttattttatagtttaaagtctttcaattcagtagtctaactgtataacatatgacttcgaacagattgatatagaccactcaatctaataatatcgttttagctacgaagagtatacaaaaaaagctagttggatagtttaaaaattaaatgctagaagaatgttggatatatcaaacaataaaattaatatttagagagaaaatatcgtaatagagttttttctataaataaatttttgagttaaaaaatattattagcctgaccaaactctttttatttctttaaactatgacagtaagatctaaattttaataaaccaatgtttatttaatattttcagaaatgggctaaaaatgactagttgttgtatgtgtttcctttgaataaaagctcaacaaacaaaaactagaagtcattatgtaaaaaaactaatgacatagtattaggtattttttgcaaattattcacacatgttgccaaaagttgttttttatattatatgtttggtcagcgaaaacggtttaaagcactctctctttcctctcgccttcctttgatgatcctttttagttcccttcttctcattgctcttgagaccatctccgaactcaaaggaaactagaaaca

SEQ ID NO: 7 - DNA, 1047 bp, Arabidopsis thaliana
aggtgcaggagattgaatcgaagcttcgtttataaaacaatgtacatagttgtttaaagtcttaaagttggacttttcttttgctcagccccacaaacccaattggattataataccgacgccgaccaatcaatgctacgaccattgacttataaaacatacatcggacacacacaaaaaaaaactacactttttgtatcagcaatctttttagttatttgttttcagttatataaatcaatgtttgtgttttctaataacagttattagtatgctagtgatgcatggttaaaaaaaaaaaaagaaatttcaaatatatattttaatttgcattatgtaaaatattttaaaaataagtccctagtaccaatggagtgtttttgtttaagctacaagtgaattataatacgtagtaatcttgattcaaatggaaagatttaccatgtgggtctataccaaaaccgtgtcctttttcatatgtatgaatctctaatagttgcttcttttgactagaataatttgtaaagtaactcctaattcataaatattgaagattagtctgatttattattaatatgcaaatctctgattaataagaaataaattataatagaaatattgatgtgtactgatcatcttttaataaatgtttgagactacaaatactataatgaatcaaatgaacataaatgtatagatgaactgaataaaattccagatacaccttgaattaaatatgtattcatagtatttttttttaaaatgaaatctggattttaaatccttattattcaaaaaaatcttctcctttgatatttttgctataacaaattaaaataaaatctgtaatcactaatcatatcccttaactccctttaatatcttttgttgaccaaaacaaatatttttttgttatatgcaacgaataaaaaaacattgaaagtactttccaaaaacaaaacacgtcttcttctccctgcggttataaatagacacggataagtacaaagaacagaggaacacgagacccaagaaaaagatctcaaa

SEQ ID NO: 8 - DNA, 1357 bp, Arabidopsis thaliana
cggtggcaaaacaattggatttttggacttggtcgcaggaagtatgatcccgttttgtcttgctcgaggttgggaaggtatgggaattgatatgattccagaggaaaagtttccagagctaaacagatggatcaaaaatttgaaagaaattgagatcgtgagagaatgtattcctcctagagaggaacaaattgagcatatgaagaaagttgtagaaagaatcaaatcagcctaagagacaacaatgatactcttcgatcagtagatgttaactatatgtttgtgtaatcttgttataatagtttgtatgaacaataaatgaaacatttgtatgaacaattagtgtttttactaaattctaatgtcaattaattattataaacattttaaatgtagtagtattcaatttcttacaaatcataagaaattggttcataaacctggagttttcatatgaaagaaaagtagataaaagctataatttagtcaaaccatccttcttaataaaaccacaactatgattaaatgtgaacatgtatttcgacacgtaagaagttgtgtaaacagaggaaagtacaacggacatgtgggtctatttgttgcggcacaatataacaaatttctttagtatatttttagtatcgtgaggtggtgagtcttgtaaccatagaaaattcaccgatactttataacattgttggaaggaaaattcagcaaacaagttcgtagatggagaaacggaaaaaccaacgcctattataacaacaacatttttaatatatgccaaaaggaacatcaaataacagattagtactaattctttttttatagctcagactatagagttgtttgttttcttgggcgtcaatataacaaacgagaatgacaaatggttttaaagctgttgatgtttcatacacagacaaaatggatcaattatgaagaaccacgataaagtgatgggtataattggggaagcagaagaaattcttgacgaagcatggggttgtcttcagaaaatgaatggatcaagtcaaggtacacatgctccagggataaggcaaggttaggaattaggacacatctccacttactaaaaaagagataaaaaaaaattataaagggaacgttataaatatgttgtaaagtcaacatctgtttccttctagactcttcgcatttacatcacactgccgaccatataaaacggcaaagttcgtcgtcgttttatcacaagaccatcaacaccataaggctataaatccaagctaaaaggtagtgattaactccacaaaaccagaaaaaactacatttctaacatatagaagaaacagagaaaaagagagagagaccccta

SEQ ID NO: 9 - DNA, 48 bp, Artificial
  primer
ggggacaagtttgtacaaaaaagcaggctccgccaactaccaacaccg

SEQ ID NO: 10 - DNA, 53 bp, Artificial
  primer
ggggaccactttgtacaagaaagctgggtaataactcttcttgtttaaatctc

SEQ ID NO: 11 - DNA, 49 bp, Artificial
  primer
ggggacaagtttgtacaaaaaagcaggctaccatggcacctgaaatacg

SEQ ID NO: 12 - DNA, 52 bp, Artificial
  primer
ggggaccactttgtacaagaaagctgggttacaaacgtccaaaacagaatcg

SEQ ID NO: 13 - DNA, 50 bp, Artificial
  primer
ggggacaagtttgtacaaaaaagcaggctgggaatctgcctcaaatatgg

SEQ ID NO: 14 - DNA, 53 bp, Artificial
  primer
ggggaccactttgtacaagaaagctgggtctcatcggaatttttcctcagagg

SEQ ID NO: 15 - DNA, 51 bp, Artificial
  primer
ggggacaagtttgtacaaaaaagcaggctcccgaacttaacccaaatgccc

SEQ ID NO: 16 - DNA, 52 bp, Artificial
  primer
ggggaccactttgtacaagaaagctgggtttgatctctaacaaaacttctcc

SEQ ID NO: 17 - DNA, 47 bp, Artificial
  primer
ggggacaagtttgtacaaaaaagcaggctcctgggacttgggctgag

SEQ ID NO: 18 - DNA, 48 bp, Artificial
  primer
ggggaccactttgtacaagaaagctgggtgtctccgccgatctggaag

SEQ ID NO: 19 - DNA, 50 bp, Artificial
  primer
ggggacaagtttgtacaaaaaagcaggctcgggttttattttggaattgg

SEQ ID NO: 20 - DNA, 54 bp, Artificial
  primer
ggggaccactttgtacaagaaagctgggttgtttctagtttcctttgagttcgg

SEQ ID NO: 21 - DNA, 49 bp, Artificial
  primer
ggggacaagtttgtacaaaaaagcaggctaggtgcaggagattgaatcg

SEQ ID NO: 22 - DNA, 54 bp, Artificial
  primer
ggggaccactttgtacaagaaagctgggttttgagatctttttcttgggtctcg

SEQ ID NO: 23 - DNA, 47 bp, Artificial
  primer
ggggacaagtttgtacaaaaaagcaggctcggtggcaaaacaattgg

SEQ ID NO: 24 - DNA, 53 bp, Artificial
  primer
ggggaccactttgtacaagaaagctgggttaggggtctctctctctttttctc